(19) United States
(12) Patent Application Publication
(10) Pub. No.: US 2006/0089681 A1
(43) Pub. Date: Apr. 27, 2006

(54) Implantable medical device

(75) Inventors: Duane Tumlinson, San Clemente, CA (US); Michael Ko, Mission Viejo, CA (US)

Correspondence Address: CROMPTON SEAGER AND TUFTE, LLC, 1221 NICOLLET AVENUE, SUITE 800, MINNEAPOLIS, MN, 55403-2420, US

(73) Assignee: Cameron Health, Inc.

(21) Appl. No.: 10/970,326

(22) Filed: Oct. 21, 2004

(51) Int. Cl. A61N 1/375

(52) U.S. Cl. 607/37

(57) ABSTRACT

An implantable medical device includes a housing and an elongate pocket formed within the housing. A locking member is positioned within the elongate pocket. A first aperture extends through the locking member in coaxial alignment with the elongate pocket, while a second aperture extends through the locking member in a direction different than that of the first aperture. A retention clip is positioned within the elongate pocket proximate the locking member and has a first configuration in which a portion of the retention clip extends into the first aperture and a second configuration in which the retention clip does not extend into the first aperture. A pushrod that has a first end that is positioned near the retention clip and a second end that is positioned exterior to the housing is disposed within the second aperture.

### TECHNICAL FIELD

The invention relates generally to implantable medical devices and more specifically to implantable medical devices that include a lead and a housing or header assembly adapted to accept the lead. In particular, the invention relates to implantable medical devices that include a housing adapted to releasably accept and secure a lead.

### BACKGROUND

A variety of implantable medical devices employ leads to provide an electrical conduit between an electrical device such as a pacemaker or a defibrillator and a portion of a patient's anatomy. In some devices, a distal end of the lead is physically secured to a portion of the patient's heart. In other devices, the distal end of the lead is positioned adjacent to the patient's heart. A proximal end of the lead is secured to the implanted electrical device. In some instances, the portion of the electrical device that secures the lead is referred to as a “header” while the remainder of the electrical device is referred to as a “can”.

There are several desired characteristics regarding the connection between the lead and the header. It can be beneficial for the lead to be securely connected to the header. It can be beneficial to gain acknowledgement that the lead is completely and properly inserted within the header, as improper or incomplete insertion can interrupt the electrical connections between the lead and the header. In some instances, improper or incomplete insertion can permit bodily fluids to enter the header and in some cases can even cause the lead to pull out of the header. In some cases, attempting to over-insert the lead can damage the lead, the header or both. In some cases, it can be desirable for the lead to be able to be removed from the header in instances such as battery replacement.

A need remains for improved connections between a lead and a header. A need also remains for a lead and header combination that provides visual, audible or tactile feedback indicating proper lead insertion and/or retention.

### SUMMARY

The invention is directed to headers that provide improved connection with leads as well as to assemblies combining a header and a lead.

Accordingly, an example embodiment of the invention can be found in an implantable medical device that includes a housing and an elongate pocket that is formed within the housing. A locking member is positioned within the elongate pocket. A first aperture extends through the locking member in axial alignment with the elongate pocket, while a second aperture extends through the locking member in a direction different than that of the first aperture.

A retention clip that is positioned within the elongate pocket near the locking member can have a first configuration in which a portion of the retention clip extends into the first aperture and a second configuration in which the retention clip does not extend into the first aperture. A pushrod having a first end positioned near the retention clip and a second end positioned exterior to the housing is disposed within the second aperture.

Another example embodiment of the invention can be found in an implantable medical device assembly that includes a lead that has an elongate shaft with a distal region, a proximal region and a proximal end. A locking recess is positioned within the proximal region of the lead. The assembly also includes a housing with an elongate pocket formed within the housing. The lead is disposed within the elongate pocket.

A locking member is positioned within the elongate pocket and includes an aperture that extends through the locking member such that the proximal region of the lead extends through the locking member. A retention clip is positioned within the elongate pocket near the locking member. The retention clip can have a first configuration in which a portion of the retention clip engages with the locking recess and a second configuration in which the retention clip does not engage with the locking recess.

An example embodiment of the invention can also be found in a method of securing a lead to a header. The lead has an elongate shaft having a distal region defined by a distal end. The header has a housing that includes an elongate pocket with a locking member disposed within the elongate pocket and a retention clip disposed near the locking member. The locking member includes an aperture axially aligned with the elongate pocket.

The lead is inserted into the elongate pocket and is advanced into the locking member until the distal end of the lead contacts the retention clip. The lead is advanced further through the locking member, thereby displacing the retention clip away from its equilibrium position. Advancing the lead further through the locking member permits the retention clip to return to its equilibrium position, or an approximation thereof.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of a header in accordance with an embodiment of the invention;

FIG. 2 is a cross-sectional view of the header of FIG. 1, taken along line 2-2;

FIG. 3 is a perspective view of a locking member in accordance with an embodiment of the invention;

FIG. 4 is a perspective view of a retention clip in accordance with an embodiment of the invention;

FIG. 5 is a perspective view of a locking member assembly including the retention clip of FIG. 4 positioned within the locking member of FIG. 3;

FIG. 6 is a perspective view of a lead in accordance with an embodiment of the invention;

FIG. 7 is a perspective view of the lead of FIG. 6 disposed within the locking member assembly of FIG. 5;

FIG. 8 is a perspective view of the lead of FIG. 6 disposed within the locking member assembly of FIG. 5, with the inclusion of a pushrod;

FIG. 9 is a cross-section of FIG. 8, taken along line 9-9, showing the retention clip in a first configuration;

FIG. 10 is a schematic cross-section in accordance with FIG. 9, showing the retention clip in a second configuration;

FIG. 11 is a perspective view of a locking member in accordance with an embodiment of the invention;

FIG. 12 is a perspective view of a retention clip in accordance with an embodiment of the invention;

FIG. 13 is a perspective view of a locking member assembly including the retention clip of FIG. 12 positioned within the locking member of FIG. 11;

FIG. 14 is a perspective view of the lead of FIG. 9 disposed within the locking member assembly of FIG. 13, with the inclusion of a pushrod;

FIG. 15 is a cross-section of FIG. 14, taken along line 14-14, showing the retention clip in a first configuration;

FIG. 16 is a schematic cross-section in accordance with FIG. 15, showing the retention clip in a second configuration;

FIG. 17 is a perspective view of an internal assembly adapted for placement within a header in accordance with an embodiment of the invention;

FIG. 18 is a perspective view of a portion of the internal assembly of FIG. 17;

FIG. 19 is a perspective view of another portion of the internal assembly of FIG. 17; and

FIG. 20 is a bottom perspective view of a housing adapted for use with the internal assembly of FIG. 17 in forming a header in accordance with an embodiment of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative but non-limiting embodiments of the claimed invention.

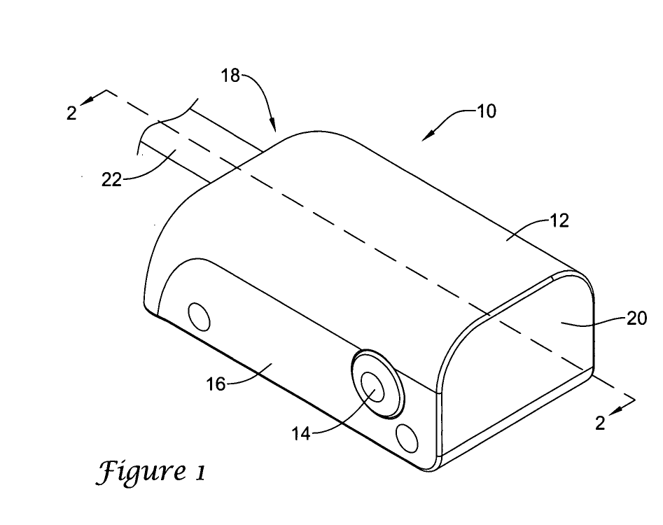

FIG. 1 illustrates a header 10 that includes a housing 12. A pushbutton 14 is positioned at a side 16 of the housing 12. The pushbutton 14 is connected to a pushrod that will be discussed in greater detail hereinafter. The housing 12 has a first end 18 and a second end 20. As illustrated, the first end 18 can correspond to an end of the header 10 that is configured to accept a lead 22.

The housing 12 can be formed of any suitable implant grade material. In some embodiments, the housing 12 or at least the second end 20 thereof can be formed of a material that is translucent or transparent. In some embodiments, only a portion of the second end 20 is formed from a translucent or transparent material. Exemplary materials include polyurethanes and epoxies.

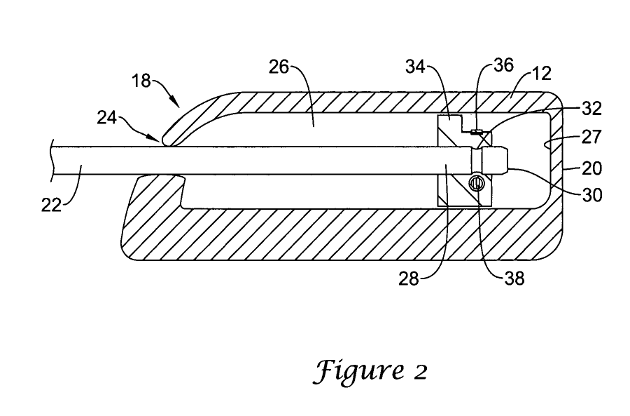

FIG. 2 is a schematic cross-sectional view of the header 10 of FIG. 1. The first end 18 of the housing 12 includes an opening 24 that leads to an elongate pocket 26. The elongate pocket 26 can have a terminus 27 at an opposite end from the opening 24. In some embodiments, the elongate pocket 26 can encompass substantially all of the interior volume of the housing 12. In other embodiments, as will be discussed in greater detail hereinafter, the interior of the housing 12 can include additional structure that itself helps to define an annulus through which the lead 22 can pass.

The lead 22 includes a proximal region 28 and a proximal end 30. A locking recess 32 is positioned within the proximal region 28 of the lead 22. In some embodiments, the locking recess 32 can be an annular groove. When the lead 22 is positioned within the header 10, the proximal region 28 of the lead 22 can extend through a locking member 34. A retention clip 36, which will be described in greater detail with respect to subsequent Figures, is positioned near the locking member 34. A pushrod 38, which also will be discussed in greater detail with respect to subsequent Figures, extends into the locking member 34.

The lead 22 can be formed of any suitable material and having any suitable dimensions. In some embodiments, the lead 22 can be formed of an electrically insulating material and can include one or more contacts that are formed of an electrically conducting material. In some embodiments, each of the one or more contacts can have a width that is in the range of about 1 mm to about 4 mm. The contacts can be separated by a distance that is in the range of about 1 mm to about 5 mm. Exemplary electrically insulating materials include polyurethane and polytetrafluoroethylene (PTFE). Exemplary electrically conducting materials include platinum, titanium and stainless steel, and alloys thereof.

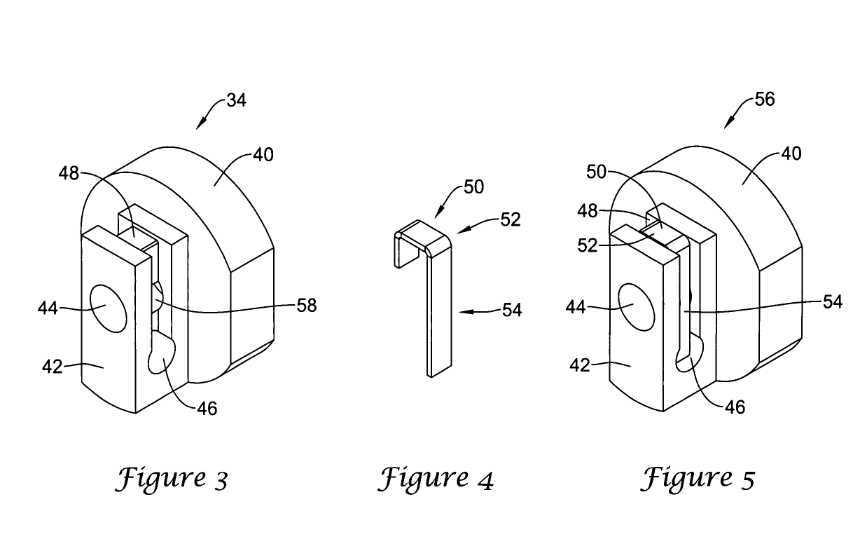

FIGS. 3 through 10 illustrate in greater detail an embodiment of the locking member 34. In particular, FIG. 3 is a perspective view of the locking member 34. The locking member 34 includes a mounting portion 40 that is configured to fit into the elongate pocket 26 (FIG. 2) and a retention portion 42 that in some embodiments can be configured to secure the retention clip 36 (FIG. 2). A first aperture 44 extends through the locking member 34. In some embodiments, as illustrated for example in FIG. 2, the lead 22 extends through the first aperture 44 when the lead 22 is disposed within the locking member 34. In some embodiments, the first aperture 44 can be considered to be axially aligned with the elongate pocket 26 (FIG. 2). In this, axial alignment is defined as the first aperture 44 and the elongate pocket 26 sharing a common axis or each having a distinct axis that is at least substantially parallel with the other.

A second aperture 46 extends through the locking member 34 in a direction that is different than that of the first aperture 44. In some embodiments, the second aperture 46 can be oriented in a direction that is at least substantially perpendicular to that of the first aperture 44. In some embodiments, the second aperture 46 can extend at a different vertical position, or elevation, than the first aperture 44.

In some embodiments, the first aperture 44 can be integrally molded or formed into the locking member 34. In other embodiments, the first aperture 44 can be drilled into the locking member 34. The first aperture 44 can have a diameter that is in the range of about 1 mm to about 4 mm. In some embodiments, the second aperture 46 can be integrally molded or formed into the locking member 34. In other embodiments, the second aperture 46 can be drilled into the locking member 34. The second aperture 46 can have a diameter that is in the range of about 0.5 mm to about 2.5 mm.

In some embodiments, the retention portion 42 of the locking member 34 includes a recessed area 48 that can be configured to accept a retention clip 50. In other embodiments, the retention portion 42 does not include the recessed area and the retention clip 50 can be configured to fit onto an exterior of the retention portion 42. FIG. 4 is a perspective view of a suitable retention clip 50 that is configured to fit within the recessed area 48 (FIG. 3). The retention clip 50 includes (in the illustrated orientation) an upper portion 52 and a straight portion 54.

In some embodiments, the upper portion 52 can be configured to fit securely into the recessed area 48 (FIG. 3). In some embodiments, the upper portion 52 can snap into the recessed area 48. In some embodiments, the upper portion 52 can have a compression fit in the recessed area 48. In other embodiments, the upper portion 52 of the retention clip 50 can be secured into the recessed area 48 using an adhesive or any suitable bonding technique.

The locking member 34 can be formed of any suitable materials including metallic and polymeric materials. In some embodiments, the locking member 34 can be formed of a metallic material. Exemplary metals for forming the locking member 34 include MP35N (implant grade stainless steels, titanium and platinum, and alloys thereof).

The retention clip 50 can be formed of any suitable materials including metallic and polymeric materials. In some embodiments, the retention clip 50 can be formed of a metallic material. Exemplary metals for forming the retention clip 50 include MP35N (implant grade stainless steels, titanium and platinum, and alloys thereof). The retention clip 50 can be formed having any suitable cross-sectional shape. As illustrated, the retention clip 50 has a rectangular cross-sectional shape with a minor dimension that is in the range of about 0.1 mm to about 1 mm and a major dimension that is in the range of about 0.25 mm to about 2 mm. In other embodiments, the retention clip 50 can have a square or ovoid cross-sectional shape.

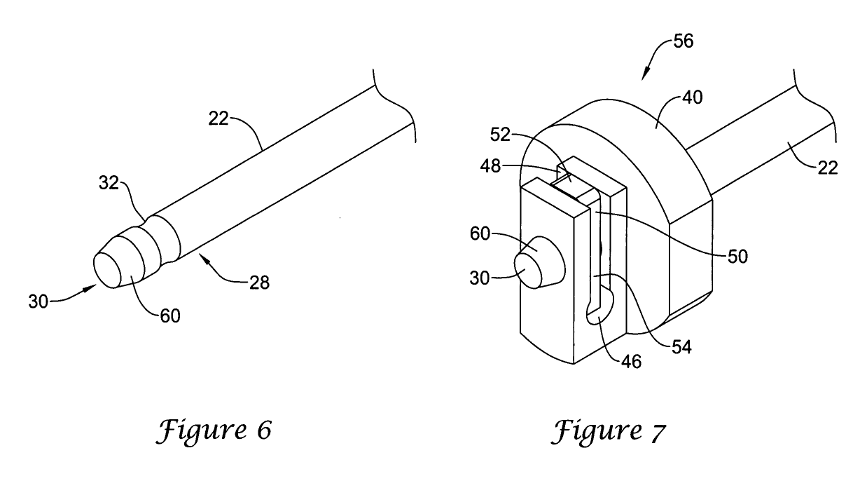

FIG. 5 illustrates a locking member assembly 56 that includes the retention clip 50 disposed within the recessed area 48 of the locking member 34. The straight portion 54 of the retention clip 50 can be seen, by comparing FIGS. 3 and 5, to extend at least partially through the first aperture 44. In particular, with reference to FIG. 3, the straight portion 54 of the retention clip 50 extends at least partially through a portion 58 of the first aperture 44. Moreover, with reference to FIG. 5, the straight portion 54 of the retention clip 50 extends downward sufficiently to extend at least partially into the second aperture 46. The interaction between the retention clip 50 and the locking member 34 provides for releasably securing the lead 22 (FIG. 2) within the header 10 (FIG. 2), as will be described in greater detail with respect to FIGS. 6 through 10.

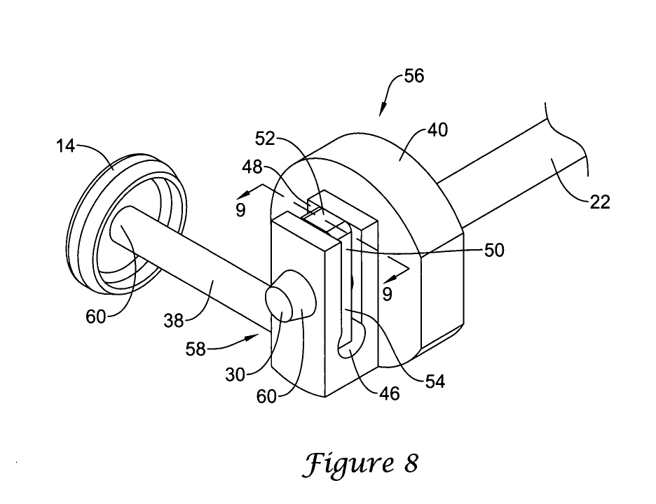

FIG. 6 is a perspective view of the lead 22, as described previously with respect to FIG. 2. In some embodiments, the proximal region 28 can include a tapered region 60. The tapered region 60 can aid in insertion of the lead 22. As illustrated in FIG. 7, the lead 22 has been positioned within the locking member assembly 56 (FIG. 5). A comparison of FIG. 5 and FIG. 7 indicates that the straight portion 54 of the retention clip, previously described as extending at least partially into the first aperture 44, consequently extends at least partially into the locking recess 32 (FIG. 6) of the lead 22.

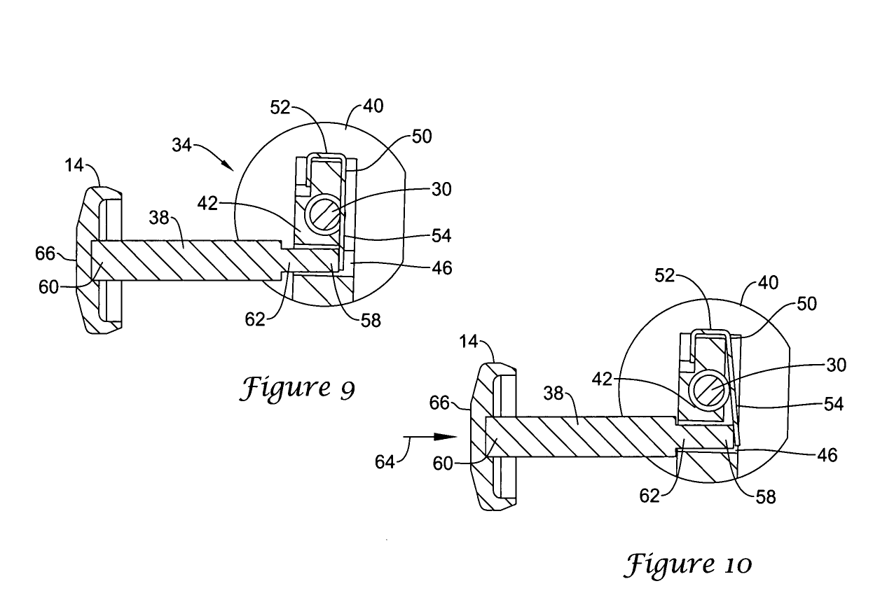

FIG. 8 is similar to FIG. 7 in that the lead 22 is seen inserted into the locking member assembly 56 (FIG. 5). In FIG. 8, however, the pushrod 38 (FIG. 2) has been positioned within the second aperture 46 (FIG. 3). In particular, the pushrod 38 has a first end 58 that is positioned within the second aperture 46 and a second end 60 that terminates at the pushbutton 14 (FIG. 1). Operation is best illustrated with reference to FIGS. 9 and 10.

FIG. 9 is a cross-sectional view of FIG. 8 illustrating a configuration that corresponds to the lead 22 being inserted into the locking member 34 and being secured therein. This configuration is also seen, for example, in FIGS. 7 and 8. The retention clip 50 is in a first configuration in which the straight portion 54 of the retention clip 50 extends at least partially into the locking recess 32 of the lead 22 (FIG. 6) and thus in some embodiments secures the lead 22 from movement with respect to the locking member 34. The retention clip 50 can be manufactured and configured such that it is biased to this first configuration.

In some embodiments, the second end 60 of the pushrod 38 is secured to the pushbutton 14. The pushbutton 14 can be secured to the pushrod 38 via a compression fitting. In some embodiments, the second end 60 of the pushrod 38 can be threaded, with complementary threading within the pushbutton 14. In other embodiments, the pushbutton 14 can be secured to the second end 60 of the pushrod 38 using an adhesive or any other suitable bonding technique. In some embodiments, the pushbutton 14 and the pushrod 38 can be integrally formed.

The pushbutton 14 and the pushrod 38 can be formed of any suitable materials and have any suitable dimensions. In some embodiments, the pushbutton 14 can be formed of any suitable polymeric or metallic material. Exemplary materials include polyacetyl and silicone rubber. The pushbutton 14 can have a diameter that is in the range of about 3 mm to about 10 mm. In some embodiments, the pushrod 38 can be formed of any suitable polymeric or metallic material. Exemplary materials include polyacetyl, titanium and stainless steel. The pushrod 38 can have a length that is in the range of about 3 mm to about 10 mm and a diameter that is in the range of about 0.5 mm to about 3 mm.

The first end 58 of the pushrod 38 is positioned next to the straight portion 54 of the retention clip 50. In the illustrated embodiment, a portion 62 of the pushrod 38 adjacent the first end 58 has a reduced diameter with respect to the rest of the pushrod 38. In some embodiments, the reduced diameter portion 62 provides a limit on inward travel of the pushrod 38. The reduced diameter portion 62 can have a length that is in the range of about 0.5 mm to about 3 mm and a diameter that is in the range of about 0.25 mm to about 2 mm. In other embodiments, the pushrod 38 can have a diameter that gradually increases or decreases between the first end 58 and the second end 60. In some embodiments, the pushrod 38 can have a diameter that remains at least substantially the same between the first end 58 and the second end 60.

As seen in FIG. 10, the lead 22 can be unlocked from the locking member 34 by applying an inward force, illustrated by arrow 64, to the pushbutton 14. As the pushrod 38 moves inward, the straight portion 54 of the retention clip 50 is moved away from the locking recess 32 (see FIG. 6) of the lead 22. This corresponds to a second configuration of the retention clip 50. With the retention clip 50 in this second configuration, the lead 22 can easily be removed and reinserted into the header 10.

In some embodiments, the lead 22 can be inserted into the elongate pocket 26 (FIG. 2) and thus into the locking member 34 while the retention clip 50 is in its second configuration, as shown in FIG. 10. A physician or other professional can simply hold the header 10 in his or her hand, with a thumb or other finger depressing the pushbutton 14. The lead 22 can be inserted until forward movement is limited by the distal end 30 of the lead 22 contacting the terminus 27 of the elongate pocket 26 (FIG. 2). At that point, he or she can simply release the pushbutton 14, which can permit the retention clip 50 to return to its first configuration and thereby lock the lead 22 into position. In some embodiments, configuring the retention clip 50 to be biased into the first configuration is sufficient in moving the retention clip 50 into the first configuration once the pushbutton 14 is released.

In some embodiments, the lead 22 can be inserted into the elongate pocket 26 (FIG. 2) and thus into the locking member 34 while the retention clip 50 is in its first configuration, as shown in FIG. 9. A physician or other professional can begin by inserting the lead 22 through the opening 24 into the elongate pocket 26 (FIG. 2). He or she can advance the lead 22 up to and into the locking member 34 until the proximal end 30 of the lead 22 contacts the retention clip 50. The lead 22 can be advanced further such that the tapered portion 60 of the lead 22 pushes against the straight portion 54 of the retention clip 50 and thus moves the retention clip 50 from its first configuration to its second configuration. The lead 22 can be advanced further such that the proximal end 30 of the lead 22 extends beyond the locking member 34 and such that the straight portion 54 of the retention clip 50 extends at least partially into the locking recess 32 (FIG. 6) of the lead 22. As a result, the retention clip 50 can return to its first configuration, and the lead 22 is thereby securely locked into position.

In some embodiments the pushbutton 14 can provide visual, tactile or audible feedback concerning the relative position of the lead 22. In some embodiments, the pushrod 38 can be biased into a position such that the first end 58 of the pushrod 38 is in contact with the straight portion 54 of the retention clip 50. As the straight portion 54 moves between its first and second configurations as described above, the pushrod 38 will similarly move inwardly and outwardly and thus can provide visual feedback regarding the relative position of the lead 22.

In particular, the pushbutton 14 can have a first position relative to the housing 12 (FIG. 1) that corresponds to the first configuration of the retention clip 50, and a second position relative to the housing 12 that corresponds to the second configuration of the retention clip. In some embodiments, the first position of the pushbutton 14 can correspond to an outer surface 66 of the pushbutton 14 being substantially flush with the housing 12, while the second position of the pushbutton 14 can correspond to the outer surface 66 of the pushbutton 14 being moved inwardly from the first position. In some embodiments, the pushbutton 14 can move a distance that is in the range of about 0.1 mm to about 1.0 mm when moving between the first position of the pushbutton 14 and the second position of the pushrod 14.

Therefore, the physician or other professional inserting the lead 22 into the header 10 can see the pushbutton 14 in its first position prior to inserting the lead 22. As the lead 22 is advanced through the locking member 34 and the retention clip 50 is forced from its first configuration to its second configuration, the pushbutton 14 will be seen to move inward. Then, as the lead 22 is advanced fully, the straight portion 54 of the retention clip 50 extends into the locking recess 32 and the retention clip 50 thus returns to its first configuration, the pushbutton 14 will be seen to move outward back to its first position.

In some embodiments, the proximal end 30 of the lead 22 can be visible through the second end 20 of the housing 12. This can provide additional visible feedback regarding the correct positioning of the lead 22.

By changing the relative vertical spacing between a point at which the straight portion 54 of the retention clip 50 contacts the locking region 32 of the lead 22 and a point at which the first end 58 of the pushrod 38 contacts the straight portion 54 of the retention clip 50, it is possible to change both the relative movement of the pushbutton 14 (as discussed above) and the force necessary to move the pushbutton 14 between its first and second positions. For example, increasing the aforementioned distance will provide for greater relative movement of the pushbutton 14 (and pushrod 38) yet will require relatively less force to move the pushrod 38 inward so as to move the retention clip 50 from its first configuration to its second configuration. Decreasing the aforementioned distance will have an opposite effect.

In some embodiments, the first end 58 of the pushrod 38 can be secured to the straight portion 54 of the retention clip 50. In some embodiments (not illustrated), the straight portion 54 of the retention clip 50 can extend through a hole or slot formed in the pushrod 38 near the first end 58 of the pushrod 38. In other embodiments, the pushrod 38 can include a biasing structure such as a spring or other similar structure positioned within the second aperture 46 in order to bias the pushrod 38 to a position proximate the retention clip 50.

In other embodiments, it is contemplated that the pushrod 38 be positioned within the housing 12 at an orientation opposite that illustrated, for example, in FIG. 8. Rather than the pushrod 38 being positioned to push against the retention clip 50 in order to move the retention clip 50 from its first configuration to its second configuration, the pushrod 38 can be positioned to pull the retention clip 50. In such embodiments, the pushrod 38 can include visible markings such as color bands (not illustrated) that can aid in providing visual feedback.

In particular, a visible marker can be placed on the exterior surface of the pushrod 38 so that the visible marker is not visible when the pushrod 38 is in a position corresponding to the retention clip 50 being in its first configuration but appears when the pushrod 38 is a position corresponding to the retention clip 50 being in its second configuration.

In some embodiments, the physician or other professional can simply hold a finger or thumb over the pushbutton 14 while inserting the lead 22 into the header 10. The physician or other professional can themselves provide a biasing force to hold the first end 58 of the pushrod 38 against the straight portion 54 of the retention clip 50 without applying sufficient force to move the retention clip 50 from its first configuration to its second configuration. As the lead 22 is advanced far enough into the locking member 34 to move the retention clip 50 into its second configuration, the physician or other professional can feel the pushbutton 14 move inward. Then, as the lead 22 is fully advanced into the locking member 34 and the retention clip 50 returns to its first configuration, the pushbutton 14 will return to its first, outermost, position. Thus, the pushbutton 14 can provide tactile feedback regarding proper insertion of the lead 22. In some embodiments, as discussed previously with respect to visual feedback, the relative positions between where the locking recess 32 (of the lead 22) and the first end 58 of the pushrod 38 contact the straight portion 54 of the retention clip 50 can influence the tactile feedback provided to the user.

In some embodiments, the locking recess 32 can be configured to provide, in combination with a sufficient biasing force that biases the retention clip 50 into its first configuration, an audible snap when the straight portion 54 of the retention clip 50 extends into the locking recess 32. Thus, audible feedback regarding the relative position of the lead 22 can be provided.

In some embodiments, the pushrod 38 and the pushbutton 14 can remain within the header 10 during and after installation of the header 10 within a patient. In some embodiments, the header 10 can include a retainer or other structure that prevents removal or inadvertent movement of the pushbutton 14 relative to the housing 12. In other embodiments, the pushbutton 14 and the pushrod 38 can be removed once the lead 22 has been successfully locked into position. A plug or seal can be inserted into the aperture within the side 16 of the housing 12 from which the pushbutton 14 and the pushrod 38 have been removed.

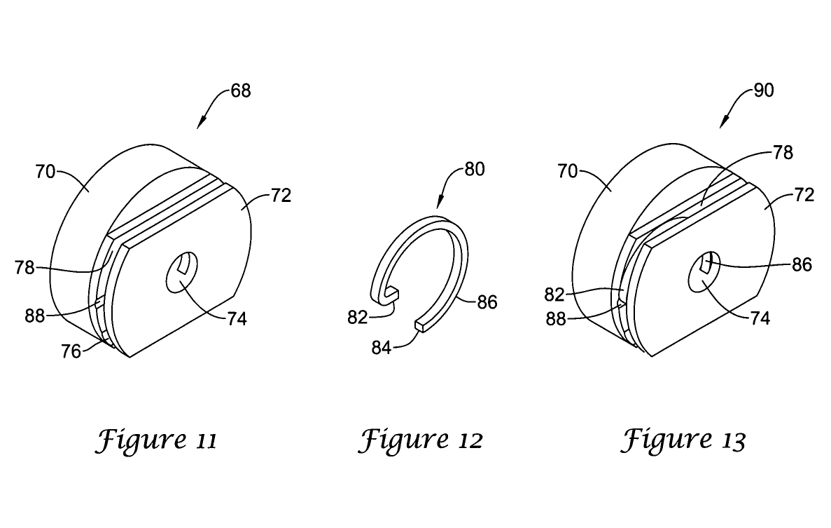

FIGS. 11-16 illustrate another embodiment of a locking member. In particular, FIG. 11 is a perspective view of a locking member 68. The locking member 68 includes a mounting portion 70 that is configured to fit into the elongate pocket 26 (FIG. 2) and a retention portion 72 that can be configured to secure another embodiment of retention clip 50 as illustrated in FIG. 12. A first aperture 74 extends through the locking member 68. In some embodiments, the lead 22 can extend through the first aperture 74 when the lead 22 is disposed within the locking member 68. In some embodiments, the first aperture 74 can be considered to be axially aligned with the elongate pocket 26 (FIG. 2).

A second aperture 76 extends through the locking member 68 in a direction that is different than that of the first aperture 74. In some embodiments, the second aperture 76 can be oriented in a direction that is at least substantially perpendicular to that of the first aperture 74. In some embodiments, the second aperture 76 can extend at a different vertical position, or elevation, than the first aperture 74.

In some embodiments, the first aperture 74 can be integrally molded or formed into the locking member 68. In other embodiments, the first aperture 74 can be drilled into the locking member 68. The first aperture 74 can have a diameter that is in the range of about 1 mm to about 4 mm. In some embodiments, the second aperture 76 can be integrally molded or formed into the locking member 68. In other embodiments, the second aperture 76 can be drilled into the locking member 68. The second aperture 76 can have a diameter that is in the range of about 0.5 mm to about 2.5 mm.

The retention portion 72 of the locking member 68 includes a recessed area 78 that can be configured to accept a retention clip. FIG. 12 is a perspective view of a suitable retention clip 80 that is configured to fit within the recessed area 78 (FIG. 11). As illustrated, the retention clip 80 is largely circular in shape, but includes a mounting portion 82, an end 84 and a curved portion 86 that extends therebetween.

In some embodiments, the retention clip 80 fits securely into the recessed area 78. The mounting portion 82 can fit into a mounting slot 88 that is positioned within the retention portion 72 of the locking member 68. In some embodiments, the mounting portion 82 can snap into the mounting slot 88. In some embodiments, the mounting portion 82 can form a compression fitting with the mounting slot 88. In other embodiments, the mounting portion 82 can be secured to the mounting slot 88 using any suitable adhesive or bonding technique.

The locking member 68 can be formed of any suitable materials including metallic and polymeric materials. In some embodiments, the locking member 68 can be formed of a metallic material. Exemplary materials for forming the locking member 68 include MP35N and steel, titanium and platinum, and alloys thereof.

The retention clip 80 can be formed of any suitable materials including metallic and polymeric materials. In some embodiments, the retention clip 80 can be formed of a metallic material. Exemplary metals for forming the retention clip 80 include MP35N and steel, titanium and platinum, and alloys thereof. The retention clip 80 can be formed having any suitable cross-sectional shape. As illustrated, the retention clip 80 has a rectangular cross-sectional shape with a minor dimension that is in the range of about 0.1 mm to about 1 mm and a major dimension that is in the range of about 0.25 mm to about 2 mm. In other embodiments, the retention clip 80 can have a square or ovoid cross-sectional shape.

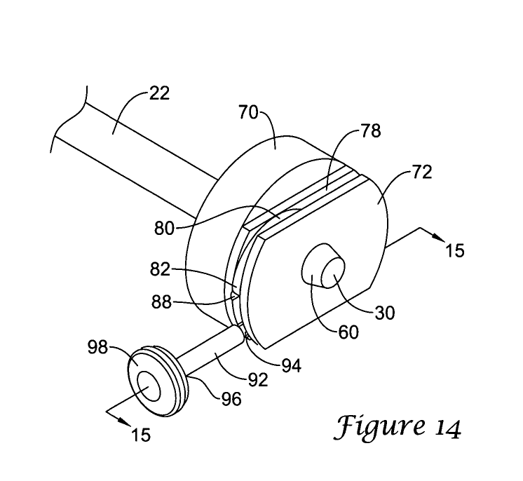

FIG. 13 illustrates a locking member assembly 90 that includes the retention clip 80 disposed within the recessed area 78 of the locking member 68. At least a portion of the curved portion 86 can be seen as extending at least partially into the first aperture 74. As with respect to the first embodiment discussed, the interaction between the retention clip 80 and the locking member 68 provides for releasably securing the lead 22 (FIG. 6), as will be discussed in greater detail with respect to FIGS. 14-16.

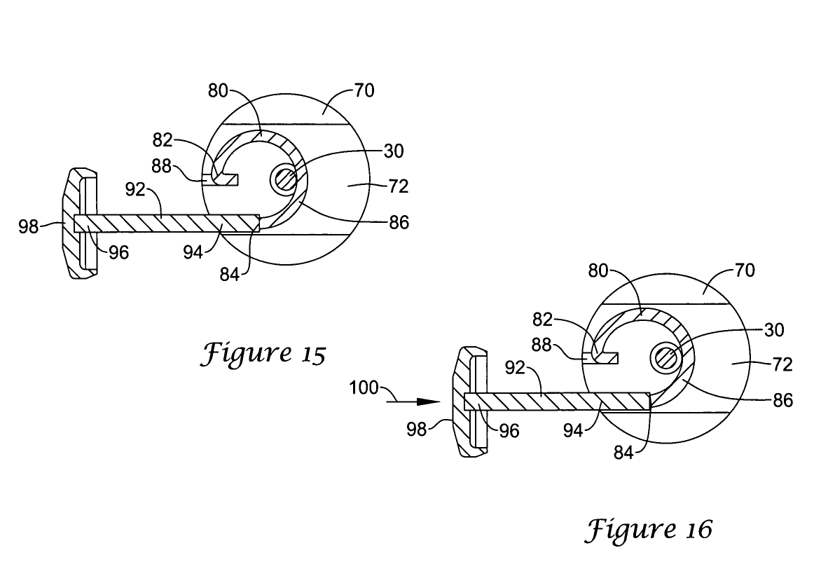

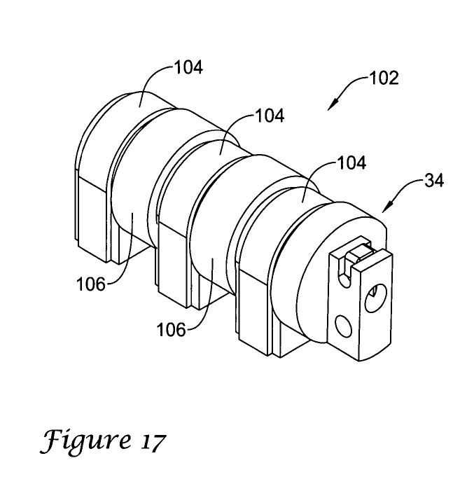

FIG. 14 is similar to FIG. 13, in that the lead 22 is seen positioned within the locking member assembly 90 (FIG. 13). In FIG. 14, however, a pushrod 92 has been positioned within the second aperture 76 (FIG. 11). In particular, the pushrod 92 has a first end 94 that is positioned within the second aperture 76 and a second end 96 that terminates in a pushbutton 98. Operation is best illustrated with reference to FIGS. 15 and 16.

FIG. 15 is a cross-sectional view of FIG. 15 illustrating a configuration that corresponds to the lead 22 being secured within the locking member 68. This configuration is also seen, for example, in FIG. 14. The retention clip 80 is in a first configuration in which the curved portion 86 extends at least partially into the locking recess 32 of the lead 22 and thus in some embodiments secures the lead 22 from movement relative to the locking member 68. The retention clip 80 can be manufactured and configured such that it is biased to this first configuration.

In some embodiments, the second end 96 of the pushrod 92 is secured to the pushbutton 98. The pushbutton 98 can be secured to the pushrod 38 via a compression fitting. In some embodiments, the second end 96 of the pushrod 92 can be threaded, with complementary threading within the pushbutton 98. In other embodiments, the pushbutton 98 can be secured to the second end 96 of the pushrod 92 using an adhesive or any suitable bonding technique. In some embodiments, the pushbutton 98 and the pushrod 92 can be integrally formed.

The pushbutton 98 and the pushrod 92 can be formed of any suitable materials and having any suitable dimensions. In some embodiments, the pushbutton 98 can be formed of any suitable polymeric or metallic material. Exemplary materials include polyacetyl and silicone rubber. The pushbutton 98 can have a diameter that is in the range of about 3 mm to about 10 mm. In some embodiments, the pushrod 92 can be formed of any suitable polymeric or metallic material. Exemplary materials include polyacetyl, stainless steel and titanium. The pushrod 92 can have a length that is in the range of about 3 mm to about 10 mm and a diameter that is in the range of about 0.5 mm to about 3 mm.

In the illustrated embodiment, the first end 94 is positioned next to the end 84 of the retention clip 80. In the illustrated embodiment, the pushrod 92 has a diameter that is at least substantially equal between the first end 94 and the second end 96. In other embodiments, as discussed with respect to the pushrod 38, the pushrod 92 can have a diameter that gradually changes between the first end 94 and the second end 96. The pushrod 92 can have a reduced diameter portion (not illustrated) that limits inward travel of the pushrod 92.

As seen in FIG. 16, the lead 22 can be unlocked from the locking member 68 by applying an inward force, illustrated by arrow 100, to the pushbutton 98. As the pushrod 92 moves inward, the curved portion 86 of the retention clip 80 is moved away from the locking recess 32 of the lead 22. This corresponds to a second configuration of the retention clip 80. With the retention clip 80 in this second configuration, the lead 22 can easily be removed and reinserted into the header 10.

While the specific geometry of the retention clip 80 and the locking member 68 varies from that of the embodiment described with respect to FIGS. 3-10, it should be noted that the embodiment illustrated in FIGS. 11-16 will function similarly with respect to releasably securing the lead 22 within the header 10. Moreover, the retention clip 80 and the locking member 68 will function similarly with respect to providing visual, audible and tactile feedback concerning the position of the lead 22 relative to the header 10.

In some embodiments, the lead 22 can be inserted into the elongate pocket 26 (FIG. 2) and thus into the locking member 76 while the retention clip 80 is in its second configuration, as shown in FIG. 16. As discussed above, a physician or other professional can depress the pushbutton 98 with his or her thumb or finger while inserting the lead 22.

In some embodiments, the lead 22 can be inserted into the header 10 while the retention clip is in its first configuration, as shown in FIG. 15, and the pushbutton 98 can provide visual, tactile or audible feedback regarding the relative position of the lead 22. In some embodiments, the pushrod 92 can be biased into a position such that the first end 94 of the pushrod 92 is in contact with the end 84 of the retention clip 80. As the retention clip 80 moves between its first and second configurations, the pushrod 92 will similarly move inward and outward and thus can provide visual confirmation of the relative position of the lead 22. Depending on the specific configuration of the locking recess 32, as discussed above, the curved portion 86 of the retention clip 80 can audibly snap into the locking recess 32, thereby providing audible feedback that the lead 22 has been properly secured.

In some embodiments, the first end 94 of the pushrod 92 can be secured to the end 84 of the retention clip 80 or the pushrod 92 can be biased into a position in which the pushrod 92 remains in contact with the retention clip 80. In other embodiments, as discussed above, a physician or other professional can provide the necessary biasing force and thus can obtain tactile feedback regarding the relative position of the lead 22.

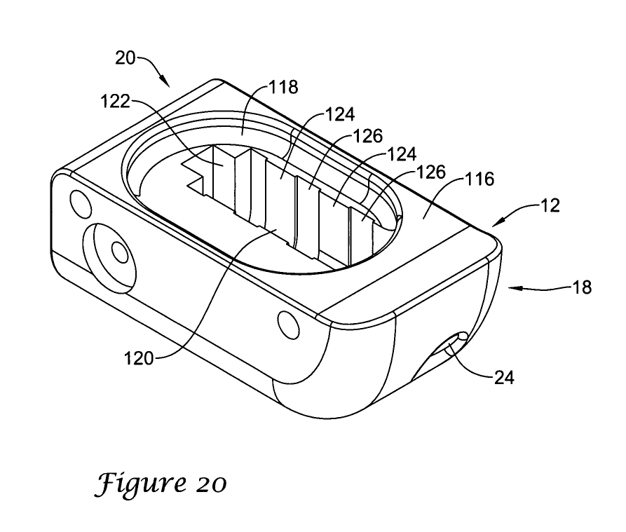

FIGS. 17-20 provide an illustrative example of forming the header 10. In particular, FIG. 17 is a perspective view of an assembly 102 that includes the locking member 34 (FIG. 3) as well as several spacer elements 104 and several conductive elements 106. In some embodiments, the assembly 102 is formed separately and is inserted as a unit into the housing 12 (as will be discussed with respect to FIG. 20). In other embodiments, each of the spacer elements 104 and the conductive elements 106 can be individually placed within the housing 12.

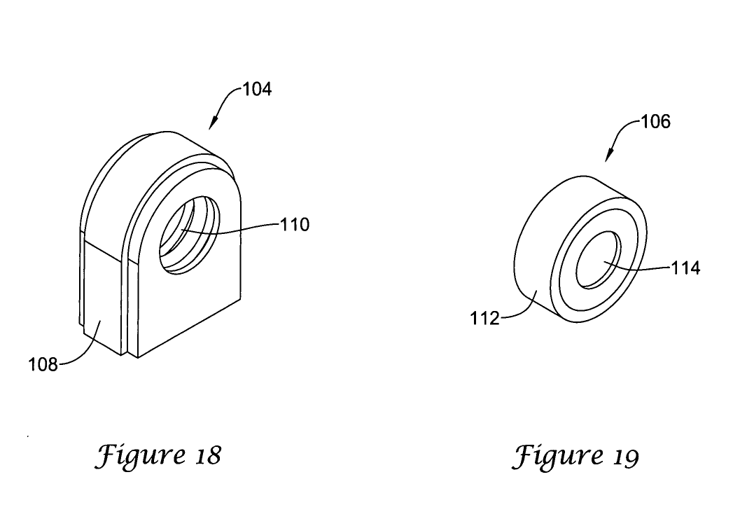

FIG. 18 is a perspective view of an illustrative embodiment of one of the spacer elements 104. The spacer element 104 includes an exterior 108 that is configured to fit into the housing 12 (as will be discussed in greater detail hereinafter) and an aperture 110 that extends through the spacer element 104. The spacer element 104 can be made from an electrically insulating material and thus can serve to electrically isolate the conductive elements 106 that can be positioned on either side of the spacer element 104 (FIG. 17). Exemplary insulating materials include silicone rubber and polyurethane.

The aperture 110 can be sized to accept the lead 22. In some embodiments, the aperture 110 can be sized and configured to accept an O-ring that is sized to accept the lead 22. It should be noted that the particular configuration shown for the spacer element 104 is intended merely as illustrative and is not intended to limit the invention in any manner.

FIG. 19 is a perspective view of an illustrative embodiment of one of the conductive elements 106. The conductive element includes an exterior 112 that is configured to fit into the housing 12 (as will be discussed in greater detail hereinafter) and an aperture 114 that extends through the conductive element 106. The conductive element 106 can be made from any suitable electrically conducting material and thus can serve to electrically communicate between the header 10 and the lead 22. Exemplary conductive materials include steel, titanium and platinum, and alloys thereof.

The aperture 114 can be sized to accept the lead 22 while retaining electrical communication therebetween. It should be noted that the particular configuration shown for the conductive element 106 is intended merely as illustrative and is not intended to limit the invention in any manner.

FIG. 20 is a bottom perspective view of the housing 12 of FIG. 1. The housing 12 includes a bottom surface 116. The bottom surface 116 can include a well 118 that is configured for mating with a can (not illustrated). The housing 12 includes an interior volume 120 that is configured to accept the assembly 102 (FIG. 17). As noted above, the assembly 102 can be inserted as a complete assembly, or each of the individual spacer elements 104, conductive elements 106 and the locking member 34 can be inserted individually.

The interior volume 120 can include portions that are configured to accept each of the above-mentioned elements. In some embodiments, the interior volume 120 can include a portion 122 that is configured to accept the locking member 34. The interior volume 120 can include one or more portions 124 that are each configured to accept a spacer element 104. The interior volume 120 can include one or more portions 126 that are each configured to accept a conductive element 106. It should be noted that the particular configuration shown for the interior volume 120, including each portion 124 and each portion 126 is intended merely to illustrate accommodation of a particular style and configuration of the spacer elements 124 and the conductive elements 126.

The assembly 102, whether inserted as a complete assembly or as a number of individual components, can be held within the interior volume 120 in any suitable manner. In some embodiments, a retaining clip or plate can be placed within the well 118. In some embodiments, the assembly 102 can be frictionally held in place. In other embodiments, the assembly 102 or each of the individual components thereof can be secured using adhesives or any other suitable bonding technology. In some embodiments, the assembly 102 can be secured in place via interaction between the header 10 and the can (not seen) to which the header 10 is secured.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What we claim is:

1. A header assembly for connecting an implantable lead to an implantable medical device, comprising:
a housing;
an elongate pocket formed within the housing;
a locking member positioned within the elongate pocket, a first aperture extending through the locking member in axial alignment with the elongate pocket, and a second aperture extending through the locking member in a direction different than that of the first aperture;
a retention clip positioned within the elongate pocket proximate the locking member, the retention clip having a first configuration in which a portion of the retention clip extends into the first aperture and a second configuration in which the retention clip does not extend into the first aperture; and
a pushrod disposed within the second aperture, the pushrod having a first end positioned proximate the retention clip and a second end positioned exterior to the housing.

2. The header assembly of claim 1, wherein the elongate pocket comprises an opening and an opposing terminus, and the locking member is positioned proximate the terminus.

3. The header assembly of claim 1, wherein the second aperture extends in a direction perpendicular to that of the first aperture.

4. The header assembly of claim 1, wherein the second aperture extends at a different elevation from the first aperture.

5. The header assembly of claim 1, wherein the retention clip is biased to the first configuration.

6. The header assembly of claim 1, wherein the locking member includes a recessed area configured to accept the retention clip.

7. The header assembly of claim 6, wherein the recessed area is configured such that the retention clip snaps into the recessed area.

8. The header assembly of claim 1, wherein the retention clip includes a straight portion that extends into the first aperture when the retention clip is in its first configuration.

9. The header assembly of claim 8, wherein the retention clip is moved from its first configuration to its second configuration by applying a force to the straight portion.

10. The header assembly of claim 8, wherein the retention clip is moved from its first configuration to its second configuration by advancing the pushrod into the housing.

11. The header assembly of claim 1, wherein the retention clip comprises a curved portion that extends into the first aperture when the retention clip is in its first configuration.

12. The header assembly of claim 11, wherein the retention clip is moved from its first configuration to its second configuration by applying a force to the curved portion.

13. The header assembly of claim 11, wherein the retention clip is moved from its first configuration to its second configuration by advancing the pushrod into the housing.

14. The header assembly of claim 1, wherein the housing proximate the terminus is transparent.

15. An implantable medical device assembly comprising:
a lead including:
an elongate shaft having a distal region, a proximal region and a proximal end with a locking recess positioned within the proximal region;
a housing having an elongate pocket formed therein, the lead disposed within the elongate pocket;
a locking member positioned within the elongate pocket, an aperture extending through the locking member such that the distal region of the lead extends through the locking member; and
a retention clip positioned within the elongate pocket proximate the locking member, the retention clip having a first configuration in which a portion of the retention clip engages with the locking recess and a second configuration in which the retention clip does not engage with the locking recess.

16. The implantable medical device assembly of claim 15, further comprising:
a second aperture extending through the locking member; and
a pushrod disposed within the second aperture, the pushrod having a first end positioned proximate the retention clip and a second end positioned exterior to the housing.

17. The implantable medical device assembly of claim 16, wherein the pushrod is disposed at least substantially perpendicular to the lead.

18. The implantable medical device assembly of claim 16, wherein the locking recess comprises an annular groove.

19. The implantable medical device assembly of claim 16, wherein the distal end of the lead is tapered.

20. The implantable medical device assembly of claim 15, wherein the retention clip is biased to the first configuration.

21. The implantable medical device assembly of claim 15, wherein the housing has a bottom surface, and the elongate pocket is open to the bottom surface.

22. The implantable medical device assembly of claim 21, further comprising an assembly positioned within the elongate pocket such that the lead extends through the assembly.

23. The implantable medical device assembly of claim 22, wherein the assembly comprises one or more annular electrodes configured to make electrical contact with the lead.

24. The implantable medical device assembly of claim 23, wherein the assembly further comprises one or more brackets disposed between the one or more annular electrodes.

25. The implantable medical device assembly of claim 22, wherein the assembly comprises components that are individually inserted into the elongate pocket.

26. The implantable medical device assembly of claim 22, wherein the assembly comprises a plurality of components that are assembled together prior to being positioned within the elongate pocket.

27. The implantable medical device assembly of claim 16, wherein the pushrod is configured to provide a visual indication of a position of the lead relative to the locking member.

28. The implantable medical device assembly of claim 27, wherein the pushrod comprises biasing means to bias the pushrod to a position corresponding to the retention clip being in its first configuration.

29. The implantable medical device assembly of claim 27, wherein the pushrod is biased such that the pushrod moves in concert with the retention clip.

30. The implantable medical device assembly of claim 29, wherein the first end of the pushrod is secured to the retention clip.

31. The implantable medical device assembly of claim 29, wherein the pushrod comprises a marker band positioned thereon such that the marker band is visible when the retention clip is in its second configuration and the marker band is not visible when the retention clip is in its first configuration.

32. A method of securing a lead to a header, the lead comprising an elongate shaft having a distal region defined by a distal end, the header including a housing comprising an elongate pocket with a locking member disposed within the elongate pocket and a retention clip disposed proximate the locking member, the locking member including an aperture axially aligned with the elongate pocket, the method comprising steps of:
inserting the lead into the elongate pocket;
advancing the lead into the locking member until the distal end of the lead contacts the retention clip;
advancing the lead further through the locking member, thereby displacing the retention clip away from an equilibrium position; and
advancing the lead further through the locking member, thereby moving the retention clip back to its equilibrium position.

33. The method of claim 32, wherein the lead comprises an annular locking groove positioned in the distal region thereof, and wherein the retention clip fits into the annular locking groove when the retention clip is in its equilibrium position.

34. The method of claim 32, wherein the locking member further comprises a second aperture, and a pushrod is positioned within the second aperture, wherein the pushrod moves with the retention clip.

35. The method of claim 34, wherein the pushrod visibly moves outwardly as the retention clip is moved away from its equilibrium position and visibly returns inwardly as the retention clip returns to its equilibrium position.

36. The method of claim 34, wherein the pushrod provides an audible indication that the retention clip has returned to its equilibrium position.
